(19) United States
(12) Patent Application Publication
(10) Pub. No.: US 2009/0318384 A1
(43) Pub. Date: Dec. 24, 2009

(54) COMPOSITION INTENDED FOR THE TREATMENT OF AMYOTROPHIC LATERAL SCLEROSIS

(75) Inventor: Michel Geffard, Talence (FR)

Correspondence Address: YOUNG & THOMPSON, 209 Madison Street, Suite 500, Alexandria, VA, 22314, US

(73) Assignee: GEMAC, Cenon (FR)

(21) Appl. No.: 12/441,808

(22) PCT Filed: Sep. 17, 2007
(86) PCT No.: PCT/FR07/51947

§ 371 Date: Mar. 18, 2009

(30) Foreign Application Priority Data
Sep. 18, 2006 (FR) 0653789

(51) Int. Cl. A61K 31/201; A61K 31/575; A61K 31/70; A61P 25/00

(52) U.S. Cl. 514/62; 514/560; 514/171

(57) ABSTRACT

The invention relates to the use of a composition for controlling the evolution of amyotrophic lateral sclerosis, characterised in that it comprises at least: a conjugate of poly-lysine and at least one anti-oxidant, and a conjugate of poly-lysine and at least one fatty acid. The invention also relates to a particular composition for controlling the evolution of amyotrophic lateral sclerosis.

This invention relates to a composition for managing the development of amyotrophic lateral sclerosis, comprising endogenic molecules that are grafted to polylysine, also called polylysine conjugates.

The invention also relates to the use of this composition.

Amyotrophic lateral sclerosis is a progressive neurodegenerative disease that is linked to the gradual alteration of motor neurons, nerve cells that control the voluntary muscles. The damage relates both to the peripheral motor neurons, in direct relation to the muscles, and the central motor neurons that are located in the motor cortex.

This systematized degeneration of the motor neurons is reflected by numerous motor problems such as the existence of spasms linked to an exaggeration of muscle tone, an increase in osteotendinous reflexes, fasciculations, or else paralyses combined with muscular atrophy. There are no other signs of neurological damage, in particular no sensory, oculomotor or “dementia” problems. Additional symptoms will nevertheless be added to the motor problems, namely: constipation, weight loss, pain, edemas, and vasomotor problems, sleeping problems and breathing problems.

According to the site where the damage of the peripheral motor neurons begins, two major forms of amyotrophic lateral sclerosis are distinguished: the spinal onset form and the bulbar onset form.

The spinal onset form is linked to the initial damage of motor neurons of the spinal cord, causing problems of motor nerve function in the upper or lower limbs.

The bulbar form is linked to the initial damage of motor neurons of the brain stem and causes problems with speaking and swallowing.

There is also a form of amyotrophic lateral sclerosis that begins with damage of the motor neurons of the motor cortex.

Regardless of the initial form, the disease always progresses toward a complete form, with multiple handicaps that could be life-threatening. In the majority of cases, death is due to a respiratory deficiency that is aggravated by a secondary bronchial infection.

Currently, there is no treatment that is able to stop the development of amyotrophic lateral sclerosis. Management of patients is limited to prevention of motor dysfunctions, assisting with handicaps, and to treatment of symptoms of the disease. In addition, this management requires the intervention of professionals and requires hospitalization and special follow-up that is burdensome for the patients.

Thus, a need persists for a treatment of amyotrophic lateral sclerosis that is able to manage the progression of the disease and is easy to administer.

This is the purpose of this invention in proposing to use a composition for managing the development of amyotrophic lateral sclerosis, comprising at least:
one conjugate between poly-lysine and at least one fatty acid, and
one conjugate between poly-lysine and at least one anti-oxidant.

The invention also proposes a particular composition that is able to manage the development of amyotrophic lateral sclerosis.

The invention is now described in detail, with regard to the accompanying figures, in which:

FIG. 1 shows the curve of the mean of normalized weights in grams of transgenic hSOD1 rats, obtained for three treatments: Dose1, Dose2 and Placebo,

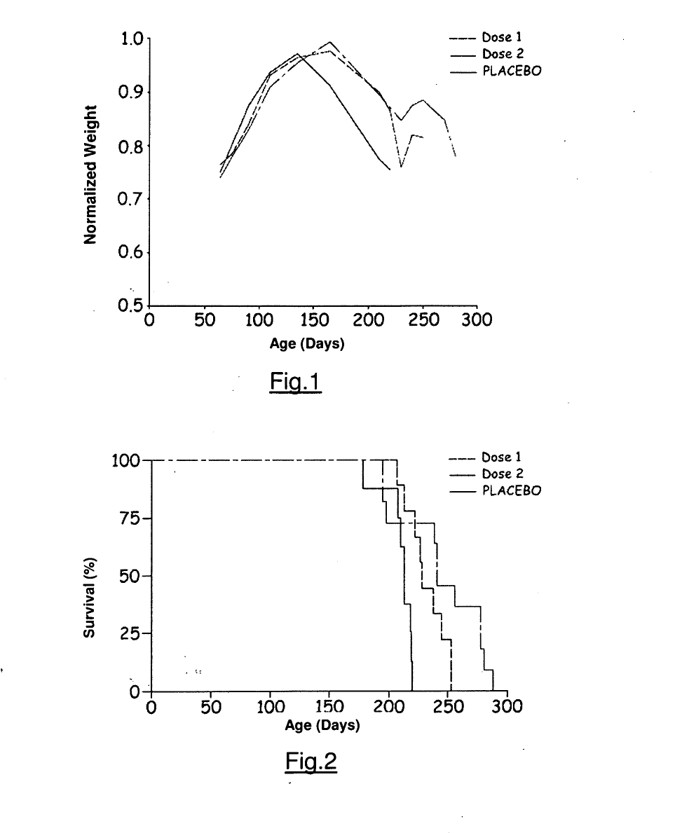

FIG. 2 shows the survival curve in days of transgenic hSOD1 rats that are treated by Dose1, Dose2 or placebo,

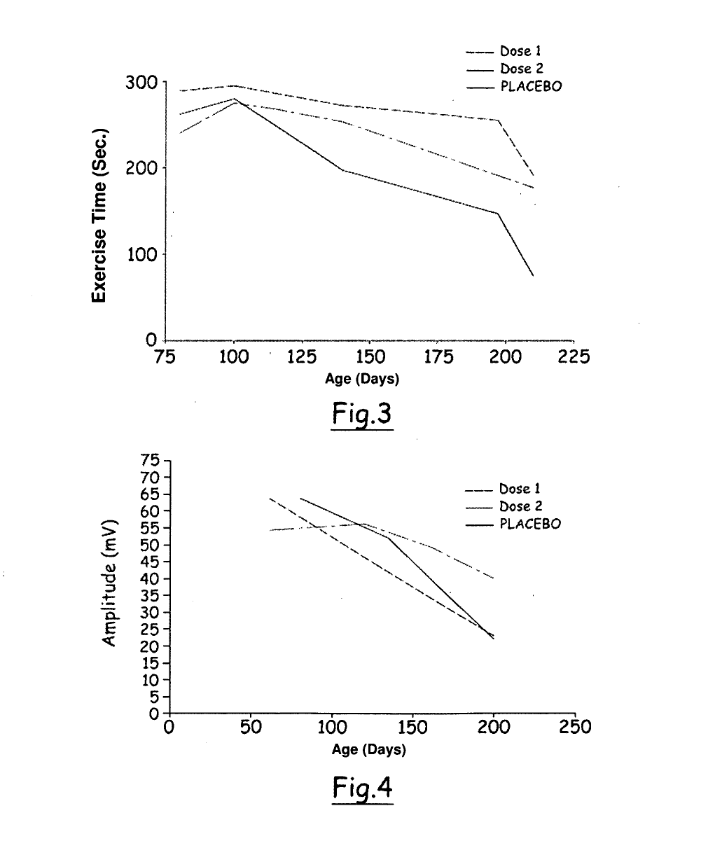

FIG. 3 shows the tracking over time of the Rotarod score (exercise time) in seconds of transgenic hSOD1 rats that are treated by Dose1, Dose2 or placebo, and

FIG. 4 shows the changes in the amplitude in mV of the above-mentioned muscle potential over time of transgenic hSOD1 rats treated by Dose1, Dose 2 or placebo.

This invention targets the use of a composition for the production of a medication that is intended to manage the development of amyotrophic lateral sclerosis, whereby the composition comprises at least:
One conjugate between poly-lysine and at least one anti-oxidant, and
One conjugate between poly-lysine and at least one fatty acid.

Anti-oxidants are defined as the known anti-oxidants and free-radical scavengers.

Such a composition makes it possible to slow down and even stop the destruction of the motor neurons and consequently to manage the progression of the disease.

According to a variant, the useful composition according to the invention comprises at least:
One conjugate between poly-lysine and at least one fatty acid,
One conjugate between poly-lysine and at least one anti-oxidant, and
One conjugate between poly-lysine and at least one amino acid derivative.

Preferably, the poly-lysine is poly-L-lysine.

To date, the mechanism at the origin of amyotrophic lateral sclerosis is not known. It is known only that sporadic forms, without any mutation, coexist with much more rare familial forms, combined with mutations at the level of the SOD1 gene that codes for the superoxide dismutase.

Although the exact origin of the pathology is not known, various hypotheses have been expressed to explain the damage of the motor neurons. These hypotheses refer to several mechanisms, in particular oxidative stress, i.e., problems of the metabolism of oxygen. Other phenomena are also suspected, such as excitotoxicity, i.e., keeping the cell in an abnormal state of excitability linked to the prolonged action of glutamate, the induction of apoptotic-type phenomena, the inadequacy of certain molecules that are necessary to the motor neurons such as the growth factors, or else the abnormal phosphorylation of neurofilaments, major components of the axonal cytoskeleton.

In individuals suffering from amyotrophic lateral sclerosis, a hyperproduction of toxic reactive oxygen radicals (ROR), in particular nitrogen monoxide (NO) and its derivatives, is noted.

Nitrogen monoxide and the ROR in general, present in a large quantity, have deleterious effects on the organism. They are responsible for modifications in the elements of the self, namely amino acids, proteins and fatty acids. They cause in particular an oxidation of thiols, fatty acids and nucleic acids of DNA, involved in the death of motor neurons.

Neuronal death would also be linked to the phenomenon of excitotoxicity that rests on the excessive mobilization of calcium in the cell under the action of an activation of receptors with glutamate. These metabolism problems of calcium induce anomalies of mitochondria that also intervene in the oxidative metabolism.

In individuals suffering from amyotrophic lateral sclerosis, there is therefore a multiplicity of very aggressive radical mechanisms that leads to the death of motor neurons.

One objective of this invention is therefore to fight against the formation of multiple free radicals that are involved in the disease and to monitor the oxidative processes that are induced so as to limit the destruction of the motor neurons.

To respond to this, the useful composition according to the invention contains a large diversity of free anti-radicals that are conjugated with poly-lysine and that trap the oxygen radicals and inhibit the pathogenic oxidative processes.

Relative to the origin of the amyotrophic lateral sclerosis and the appearance of the oxidative processes, it is assumed that the pathology is linked to an unknown external causative agent. This bacterial or viral environmental stress would trigger the production of antibodies against the components of these bacteria or viruses. The induced immune response would be involved in the death of the motor neurons. Among the antibodies that circulate in the patients suffering from amyotrophic lateral sclerosis, there are in particular antibodies that are directed against short-chain fatty acids.

This is why the composition according to the invention contains fatty acids that are conjugated with poly-lysine, in particular short-chain fatty acids that play a decoy role for the short-chain fatty acids that are carried by the potential causative agent of amyotrophic lateral sclerosis.

Although the initial factor of the disease is not known, it is known that amyotrophic lateral sclerosis is a multi-factor disease that is linked to a certain number of identified mechanisms.

One objective of the invention is therefore to act on these various mechanisms and to combine the actions.

Actually, the composition according to the invention contains at least two types of endogenic molecules that have complementary and combined actions that make it possible to act on the various aspects of amyotrophic lateral sclerosis.

According to a variant, the composition according to the invention contains at least three types of endogenic molecules.

To be effective, the molecules of the composition according to the invention cannot be used unlinked because they would be quickly metabolized, would not reach their target, and would not have any therapeutic activity.

Actually, according to the invention, these endogenic molecules are grafted to a particular vector: poly-lysine. This particular vector makes it possible:
To avoid the metabolic degradation of the endogenic molecules,
To reach their targets with endogenic molecules, and
To provide a therapeutic activity to the endogenic molecules that they do not have without this grafting.

Furthermore, poly-lysine also has the advantage of being inert, non-allergenic, non-immunogenic, and of having a rather long half-life.

Advantageously, the composition according to the invention contains only endogenic substances, i.e., known to be present naturally in the living. It has neither toxicity nor secondary effects and can be administered over the long term.

The composition according to the invention can be incorporated in various types of pharmaceutical preparations that are presented in all galenical forms.

Among the galenical forms of pharmaceutical preparations that can include the active ingredient according to this invention, it is possible to cite in particular the sublingual form, which is practical to use and equivalent in effectiveness to subcutaneous administration.

Among the compositions according to the invention, it is possible to cite a particular composition that contains the following poly-L-lysine conjugates:
Oleic acid—poly-L-lysine—thioctic acid
Oleic acid—poly-L-lysine—myristic acid
Oleic acid—poly-L-lysine—palmitic acid
Oleic acid—poly-L-lysine—lauric acid
Oleic acid—poly-L-lysine—linoleic acid
Oleic acid—poly-L-lysine—palmitoleic acid
Oleic acid—poly-L-lysine—caprylic acid
Trans, trans-framesyl-L-cysteine—poly-L-lysin—palmitic acid
Cholesterol—poly-L-lysine—oleic acid
L-Cysteine—reduced glutaric aldehyde—poly-L-lysine
L-Cysteine—glutaric anhydride—poly-L-lysine
Taurine—reduced glutaric aldehyde—poly-L-lysine
Taurine—glutaric anhydride—poly-L-lysine
L-Methionine—reduced glutaric aldehyde—poly-L-lysine
L-Methionine—glutaric anhydride—poly-L-lysine
L-Glutathione—reduced glutaric aldehdye—poly-L-lysine
Alpha-tocopherol succinate—poly-L-lysine
Ascorbic acid—poly-L-lysine
Oleic acid—poly-L-lysine—coenzymeQ10
Oleic acid—poly-L-lysine—retinoic acid
Pantothenic acid—poly-L-lysine—oleic acid
Biotin—poly-L-lysine
Uric acid—formaldehyde—poly-L-lysine
Agmatine—reduced glutaric aldehyde—poly-L-lysine
Glucosamine—glutaric anhydride—poly-L-lysine

The effectiveness on the management of the development of amyotrophic lateral sclerosis of this composition has been tested on the single animal model known for this pathology, the model of transgenic SOD1 rats and mice.

The study was carried out on 29 transgenic rats that overexpress the hSOD1 gene, developing amyotrophic lateral sclerosis symptoms of the familial form.

The operating procedure consists in subcutaneously injecting 500 μl of the composition according to the invention, Dose1 or Dose2 or a placebo, daily from D65 to D185.

The concentrations of M (mol/liter) of the elements of the composition for Dose1 are as follows:

| | |
|---|---|
| Oleic acid - poly-L-lysine - thioctic acid | 3.3 · 10⁻⁵ |
| Oleic acid - poly-L-lysine - myristic acid | 3.3 · 10⁻⁵ |
| Oleic acid - poly-L-lysine - palmitic acid | 3.3 · 10⁻⁵ |
| Oleic acid - poly-L-lysine - lauric acid | 3.3 · 10⁻⁵ |
| Oleic acid - poly-L-lysine - linoleic acid | 3.3 · 10⁻⁵ |
| Oleic acid - poly-L-lysine - palmitoleic acid | 3.3 · 10⁻⁵ |
| Oleic acid - poly-L-lysine - caprylic acid | 3.3 · 10⁻⁵ |
| Trans, trans-farnesyl-L-cysteine - poly-L-lysine - palmitic acid | 3.3 · 10⁻⁵ |
| Cholesterol - poly-L-lysine - oleic acid | 3.3 · 10⁻⁵ |
| L-Cysteine - reduced glutaric aldehyde - poly-L-lysine | 3.3 · 10⁻⁵ |
| L-Cysteine - glutaric anhydride - poly-L-lysine | 3.3 · 10⁻⁵ |
| Taurine - reduced glutaric aldehyde - poly-L-lysine | 3.3 · 10⁻⁵ |
| Taurine - glutaric anhydride - poly-L-lysine | 3.3 · 10⁻⁵ |
| L-Methionine - reduced glutaric aldehyde - poly-L-lysine | 3.3 · 10⁻⁵ |
| L-Methionine - glutaric anhydride - poly-L-lysine | 3.3 · 10⁻⁵ |
| L-Glutathione - reduced glutaric aldehyde - poly-L-lysine | 3.3 · 10⁻⁵ |
| Alpha-tocopherol succinate - poly-L-lysine | 1 · 10⁻⁵ |
| Ascorbic acid - poly-L-lysine | 1 · 10⁻⁵ |
| Oleic acid - poly-L-lysine - coenzymeQ10 | 1 · 10⁻⁵ |
| Oleic acid - poly-L-lysine - retinoic acid | 1 · 10⁻⁵ |
| Pantothenic acid - poly-L-lysine - oleic acid | 1 · 10⁻⁵ |
| Biotin - poly-L-lysine | 1 · 10⁻⁵ |
| Uric acid - formaldehyde - poly-L-lysine | 3.3 · 10⁻⁵ |
| Agmatine - reduced glutaric aldehyde - poly-L-lysine | 3.3 · 10⁻⁵ |
| Glucosamine - glutaric anhydride - poly-L-lysine | 3.3 · 10⁻⁵ |

The M concentrations (mol/liter) of the elements of the composition for Dose2 are as follows:

| | |
|---|---|
| Oleic acid - poly-L-lysine - thioctic acid | 1 · 10⁻⁴ |
| Oleic acid - poly-L-lysine - myristic acid | 1 · 10⁻⁴ |
| Oleic acid - poly-L-lysine - palmitic acid | 1 · 10⁻⁴ |
| Oleic acid - poly-L-lysine - lauric acid | 1 · 10⁻⁴ |
| Oleic acid - poly-L-lysine - linoleic acid | 1 · 10⁻⁴ |
| Oleic acid - poly-L-lysine - palmitoleic acid | 1 · 10⁻⁴ |
| Oleic acid - poly-L-lysine - caprylic acid | 1 · 10⁻⁴ |
| Trans, trans-farnesyl-L-cysteine - poly-L-lysine - palmitic acid | 1 · 10⁻⁴ |
| Cholesterol - poly-L-lysine - oleic acid | 1 · 10⁻⁴ |
| L-Cysteine - reduced glutaric aldehyde - poly-L-lysine | 1 · 10⁻⁴ |
| L-Cysteine - glutaric anhydride - poly-L-lysine | 1 · 10⁻⁴ |
| Taurine - reduced glutaric aldehyde - poly-L-lysine | 1 · 10⁻⁴ |
| Taurine - glutaric anhydride - poly-L-lysine | 1 · 10⁻⁴ |
| L-Methionine - reduced glutaric aldehyde - poly-L-lysine | 1 · 10⁻⁴ |
| L-Methionine - glutaric anhydride - poly-L-lysine | 1 · 10⁻⁴ |
| L-Glutathione - reduced glutaric aldehyde - poly-L-lysine | 1 · 10⁻⁴ |
| Alpha-tocopherol succinate - poly-L-lysine | 3 · 10⁻⁵ |
| Ascorbic acid - poly-L-lysine | 3 · 10⁻⁵ |
| Oleic acid - poly-L-lysine - coenzymeQ10 | 3 · 10⁻⁵ |
| Oleic acid - poly-L-lysine - retinoic acid | 3 · 10⁻⁵ |
| Pantothenic acid - poly-L-lysine - oleic acid | 3 · 10⁻⁵ |
| Biotin - poly-L-lysine | 3 · 10⁻⁵ |
| Uric acid - formaldehyde - poly-L-lysine | 1 · 10⁻⁴ |
| Agmatine - reduced glutaric aldehyde - poly-L-lysine | 1 · 10⁻⁴ |
| Glucosamine - glutaric anhydride - poly-L-lysine | 1 · 10⁻⁴ |

On D65, the rats are aged approximately 65 days.

Several parameters are evaluated at regular intervals.

Blood samples are taken by venipuncture at the rat's tail on D60, D90, D 115, D140, D225 and at the time of sacrifice at the end of the experiment.

The weight of the animals is listed at least once per week. The weight curve makes it possible to detect directly whether an animal develops muscular atrophy and therefore a loss of mass.

The curve of the mean of the normalized weights obtained for each of the three treatments is shown in FIG. 1. It is noted that the animals that are treated with the composition according to the invention (Dose1, Dose2) have a weight curve that is improved relative to the animals that are treated by the placebo.

Furthermore, the survival of the animals is observed during the procedure.

The survival curve according to the treatment, presented in FIG. 2, shows that the survival rate of the rats that are treated with the composition according to the invention (Dose1, Dose2) is increased significantly relative to that of the animals treated by placebo. In addition, the mean survival time of the animals that are treated with the placebo is 210 days, whereas that of the animals treated with Dose 1 is 230 days and that of the animals treated with Dose 2 is 248 days.

Tests that make it possible to evaluate the driving capacity of the animals are also carried out on a device that is equipped with a rotating bar (Rotarod).

The tracking over time of the Rotarod score (exercise time expressed in seconds) according to the treatment is shown in FIG. 3. In addition, the results of the Rotarod score obtained on day 210, expressed in seconds according to the treatment, are presented in the table below:

| | Placebo | Dose 1 | Dose 2 |
|---|---|---|---|
| Exercise Time(s) | 75 | 180 | 170 |

These results show that the rats that are treated by the composition according to the invention have a better driving function.

Finally, electromyographic tests are carried out on each animal over several periods: before the inclusion in the procedure and during the procedures on D140 and D200. Several parameters are measured, in particular the amplitude of the above-mentioned muscle potential in the muscles of the anterior tibial compartment.

The results that are presented in FIG. 4 show that the rats that are treated with the composition according to the invention with Dose2 maintain, on day 200, an amplitude that is greater than that of the animals treated by placebo.

In a general manner, this study shows that the composition according to the invention has a beneficial effect on the transgenic hSOD1 rats, model of amyotrophic lateral sclerosis.

Of course, the invention obviously is not limited to the use of this sample composition that is shown and described above, but on the contrary covers all of its variants, in particular relative to fatty acids, anti-oxidants, and derivatives of amino acids that are used, as well as the preparations that can include the composition.

1-5. (canceled)

6. A composition to manage the development of amyotrophic lateral sclerosis, comprising:
at least one conjugate between poly-lysine and at least one anti-oxidant, and
at least one conjugate between poly-lysine and at least one fatty acid.

7. The composition according to claim 6, further comprising:
at least one conjugate between poly-lysine and at least one amino acid derivative.

8. The composition according to claim 6, wherein the composition is able to trap reactive oxygen radicals.

9. The composition according to claim 7, wherein the poly-lysine is poly-L-lysine.

10. The composition according to claim 9, wherein the poly-L-lysine conjugates are:
Oleic acid—poly-L-lysine—thioctic acid,
Oleic acid—poly-L-lysine—myristic acid,
Oleic acid—poly-L-lysine—palmitic acid,
Oleic acid—poly-L-lysine—lauric acid,
Oleic acid—poly-L-lysine—linoleic acid,
Oleic acid—poly-L-lysine—palmitoleic acid,
Oleic acid—poly-L-lysine—caprylic acid,
Trans, trans-farnesyl-L-cysteine—poly-L-lysine—palmitic acid,
Cholesterol—poly-L-lysine—oleic acid,
L-Cysteine—reduced glutaric aldehyde—poly-L-lysine,
L-Cysteine—glutaric anhydride—poly-L-lysine,
Taurine—reduced glutaric aldehyde—poly-L-lysine,
Taurine—glutaric anhydride—poly-L-lysine,
L-Methionine—reduced glutaric aldehyde—poly-L-lysine,
L-Methionine—glutaric anhydride—poly-L-lysine,
L-Glutathione—reduced glutaric aldehdye—poly-L-lysine,
Alpha-tocopherol succinate—poly-L-lysine,
Ascorbic acid—poly-L-lysine,
Oleic acid—poly-L-lysine—coenzymeQ10,
Oleic acid—poly-L-lysine—retinoic acid,
Pantothenic acid—poly-L-lysine—oleic acid,
Biotin—poly-L-lysine,
Uric acid—formaldehyde—poly-L-lysine,
Agmatine—reduced glutaric aldehyde—poly-L-lysine, and
Glucosamine—glutaric anhydride—poly-L-lysine.

11. The composition according to claim 6, further comprising a pharmaceutically acceptable carrier.

12. A method of treating amyotophic lateral sclerosis, comprising administering a composition comprising an effective amount of at least one conjugate between poly-lysine and at least one anti-oxidant, and at least one conjugate between poly-lysine and at least one fatty acid to a subject in need thereof.

13. The method according to claim 12, wherein the composition further comprises at least one conjugate between poly-lysine and at least one amino acid derivative.

14. The method according to claim 12, wherein the composition traps reactive oxygen radicals.

15. The method according to claim 13, wherein the poly-lysine is poly-L-lysine.

16. The method according to claim 15, wherein the poly-L-lysine conjugates are:
Oleic acid—poly-L-lysine—thioctic acid,
Oleic acid—poly-L-lysine—myristic acid,
Oleic acid—poly-L-lysine—palmitic acid,
Oleic acid—poly-L-lysine—lauric acid,
Oleic acid—poly-L-lysine—linoleic acid,
Oleic acid—poly-L-lysine—palmitoleic acid,
Oleic acid—poly-L-lysine—caprylic acid,
Trans, trans-farnesyl-L-cysteine—poly-L-lysine—palmitic acid,
Cholesterol—poly-L-lysine—oleic acid,
L-Cysteine—reduced glutaric aldehyde—poly-L-lysine,
L-Cysteine—glutaric anhydride—poly-L-lysine,
Taurine—reduced glutaric aldehyde—poly-L-lysine,
Taurine—glutaric anhydride—poly-L-lysine,
L-Methionine—reduced glutaric aldehyde—poly-L-lysine,
L-Methionine—glutaric anhydride—poly-L-lysine,
L-Glutathione—reduced glutaric aldehdye—poly-L-lysine,
Alpha-tocopherol succinate—poly-L-lysine,
Ascorbic acid—poly-L-lysine,
Oleic acid—poly-L-lysine—coenzymeQ10,
Oleic acid—poly-L-lysine—retinoic acid,
Pantothenic acid—poly-L-lysine—oleic acid,
Biotin—poly-L-lysine,
Uric acid—formaldehyde—poly-L-lysine,
Agmatine—reduced glutaric aldehyde—poly-L-lysine, and
Glucosamine—glutaric anhydride—poly-L-lysine.

17. The method according to claim 12, wherein the composition further comprises a pharmaceutically acceptable carrier.
